(19) United States
(12) Patent Application Publication
(10) Pub. No.: US 2019/0281862 A1
(43) Pub. Date: Sep. 19, 2019

(54) METHOD FOR ENHANCING OMEGA-3 OIL CONTENT IN LIVESTOCK

(71) Applicant: Avocet IP Limited, Berwick Upon Tweed, Northumberland (GB)

(72) Inventor: James Robert JENNINGS, Yarm, North Yorkshire (GB)

(21) Appl. No.: 16/466,121

(22) PCT Filed: Dec. 4, 2017
(86) PCT No.: PCT/EP2017/025353

§ 371 Date: Jun. 3, 2019

(30) Foreign Application Priority Data
Dec. 2, 2016 (GB) 1620560.1

(51) Int. Cl. A23K 10/30; A23K 50/10; A23B 9/22; A01G 31/02; C12M 1/107

(52) U.S. Cl. CPC A23K 10/30; A23K 50/10; C12M 21/04; A01G 31/02; A23B 9/22

(57) ABSTRACT

A method for enhancing Omega-3 oil content in body tissue of livestock include operating a livestock feed production arrangement for generating feed for livestock. The livestock feed production arrangement includes an arrangement for supplying seeds, a hydroponics arrangement for receiving the supplied seeds and for germinating and growing the supplied seed in a light-excluded environment in one or more hydroponics trays to generate plant growth material, and a harvesting arrangement for receiving the plant growth material and processing the plant growth material to generate the corresponding livestock feed for livestock. The method also includes feeding the livestock feed to livestock of a variety to enhance the Omega-3 oil content in their body tissue.

### TECHNICAL FIELD

The present disclosure generally relates to enhancing quality of livestock, and more specifically to a method for enhancing Omega-3 oil content in body tissue of livestock. Moreover, the present disclosure relates to computer program products comprising a non-transitory computer-readable storage medium having computer-readable instructions stored thereon, the computer-readable instructions being executable by a computerized device comprising processing hardware to execute aforesaid methods.

### BACKGROUND

It is well known that Omega-3 oils are powerful antioxidants and are highly beneficial for achieving a good state of health. In contemporary times, fish oils have been a relatively inexpensive source of Omega-3 oils, for example salmon oil and cod oil. However, with severe over-fishing presently occurring, such that many fish stocks are not managed in a sustainable manner and will decline steadily in future, fish-derived Omega-3 oils will become expensive in future. Furthermore, with pollution in ocean environments steadily increasing as a result of human activities, for example resulting from radiation discharges from Sellafield, La Hague and Fukushima Daiichi, there are also growing concerns about safety of consuming fish-oils in future, as aquatic pollutants become incorporated into fish tissue, for example Plutonium-239, Strontium-90, Caesium 134 and Caesium 139, and similar. Thus, from the foregoing, it will be appreciated that alternative sources of Omega-3 oils are desirable to develop.

Present farming techniques for rearing cattle are not sufficiently productive to meet future demand for meat products at a cost that future population are likely to be able to afford, as energy-per-capita (e) falls drastically as a consequence of increasing World population (presently 7 billion people) and depletion of fossil fuel reserves. As a result, there arises a technical problem of how to make agriculture more efficient and productive, especially in respect of meat production, as energy-per-capita (e) falls, and also able to satisfy requirements for supply of Omega-3 oils for enhancing human health.

Livestock is typically conventionally allowed to graze in pasture and similar terrain, and fed on silage in winter, wherein the silage is generated from harvested plant material, by way of a fermentation process. The silage provides the livestock with sufficient nutrition to survive, it is not particularly exciting for the livestock to eat, that enjoy fresh plant material with exciting aroma and texture; this is important with regards to animal welfare.

Although intensively farmed animals are known, for example as in poultry industry, such techniques are not suitable for other types of animals than poultry. Therefore, there arises a need to provide improved livestock feed production apparatus for supporting advanced highly productive forms of agriculture. Of particular importance is to ensure that cattle receive sufficient roughage for their digestive systems to function in an efficient manner, which is also conducive to good animal welfare.

Conventionally, cattle on farms are grazed on grass in an outdoor environment and are provided with food supplements on a periodic basis, for example during severe winter months when the cattle are settled indoors. Moreover, there is an increasing demand for protein products worldwide, especially those high in antioxidants such as Omega-3 oils that are often desired to be derived from fish sources. With drastic reductions in fish stocks due to overfishing and aquatic pollution due to discharges from industry, nuclear power plants (e.g. Fukushima Daiichi, ruined in year 2011) and discharges from coastal urbanized regions, there arises a need to feed human population with sources of Omega-3 oils other than fish sources.

### SUMMARY

The present disclosure seeks to provide a method for enhancing Omega-3 oil content in body tissue of livestock.

In one aspect, an embodiment of the present disclosure provides a method for enhancing Omega-3 oil content in body tissue of livestock, the method comprises:

(i) operating a livestock feed production arrangement for generating feed for livestock, wherein the livestock feed production arrangement includes an arrangement for supplying seeds, a hydroponics arrangement for receiving the supplied seeds and for germinating and growing the supplied seed in a light-excluded environment in one or more hydroponics trays to generate plant growth material, and a harvesting arrangement for receiving the plant growth material and processing the plant growth material to generate the corresponding livestock feed for livestock; and
(ii) feeding the livestock feed to livestock of a variety to enhance the Omega-3 oil content in their body tissue.

The present disclosure provides a method for enhancing quality of livestock, particularly, a meat quality of the livestock. Specifically, the present disclosure relates to enhancing Omega-3 oil content in body tissue of livestock, i.e. to produce livestock employing, for example, Piedmontese or similar types of cattle that include a relatively higher concentration of Omega-3 oils in their body tissue. The present disclosure is advantageous on the account that the system for enhancing the Omega-3 content in the body tissue of cattle promotes the overall welfare of the livestock. Further, the present disclosure enables in increasing a digestive efficiency of the livestock by providing sufficient roughage in the form of supplementary materials to the fodder, and further aiding the overall health of the livestock. Moreover, the present disclosure is of the advantage that a nutritious feed improves a recovery time against day to day work for the livestock.

Optionally, the method includes that the variety of the livestock comprises Piedmontese.

Optionally, the method includes arranging for the cattle to undergo 3 pregnancies within a 2-year period by use of artificial insemination, embryo implanting and varying a diet of the plant growth material in coordination with the pregnancies.

Optionally, the method includes operating the livestock feed production arrangement to blend supplementary materials into the corresponding livestock feed.

Optionally, the livestock feed production arrangement is operable to blend the supplementary materials into the corresponding livestock feed in a temporally varying manner.

Optionally, the method includes arranging for the supplementary materials to include at least one of hay, wild flower supplements, aromatic supplements, vitamin supplements, growth supplements, antibiotics.

Optionally, the method includes employing a gaseous fumigation arrangement for gaseously fumigating the supplied seed prior to germination to reduce an occurrence of mould growth therein during germination and growing of the seeds to form roots and shoots.

Optionally, the method includes operating the gaseous fumigation arrangement to employ ozone gas for fumigating the supplied grain to reduce growth of mould in the plant growth material.

Optionally, the method includes providing the ozone gas for fumigation in a concentration in a range of 20 to 40 parts per million (ppm) for a period in a range of 5 minutes to 1 hour.

Optionally, the method includes providing the ozone gas for fumigation temporally periodically for fumigating the supplied grain in response to detection of mould formation upon the supplied seeds, their roots and/or their shoots.

Optionally, the method includes applying the ozone gas in combination with sulphur dioxide and/or chlorine gas.

Optionally, the method includes arranging for the one of more hydroponics trays have a length:width ratio in a range of 3:1 to 10:1.

Optionally, the method includes arranging for the one or more hydroponics trays to be provided with an actuation arrangement for selectively flexing regions of the one or more hydroponics trays for sweeping ponding of nutrient solution occurring therein along a length of the one or more hydroponics trays.

Optionally, the method includes arranging for the one or more hydroponics trays to be disposed in operation on a carousel for at least one of: applying seeds to the one or more hydroponics trays for growing the plant growth material on the one or more hydroponics trays, harvesting the plant growth material from the one or more hydroponics trays.

Optionally, the method includes arranging for the one or more hydroponics trays to be disposed in one or more vertical stacks, wherein planes of the one or more hydroponics trays for receiving the grain are mutually parallel.

According to another embodiment, the present disclosure provides an agricultural system comprising the livestock feed production arrangement and a housing arrangement for accommodating livestock, wherein the livestock is fed in operation from the livestock feed production arrangement, and the livestock feed production arrangement is operated according to the method disclosed herein above.

Optionally, the livestock feed production apparatus includes an arrangement for receiving waste from the animals and, for anaerobically digesting the waste to generate methane gas.

Optionally, the agricultural system is operable to employ, the method disclosed herein above, off-grid with electricity supplied from at least one of: methane fuel, solar cells, wind turbines, naturally-occurring water flow.

According to yet another embodiment, the present disclosure provides a computer program product comprising a non-transitory computer-readable storage medium having computer-readable instructions stored thereon, the computer-readable instructions being executable by a computerized device comprising processing hardware to execute the method disclosed herein above.

It will be appreciated that features of the invention are susceptible to being combined in various combinations without departing from the scope of the invention as defined by the appended claims.

### DESCRIPTION OF THE DIAGRAMS

Embodiments of the present invention will now be described, by way of example only, with reference to the following diagrams wherein:

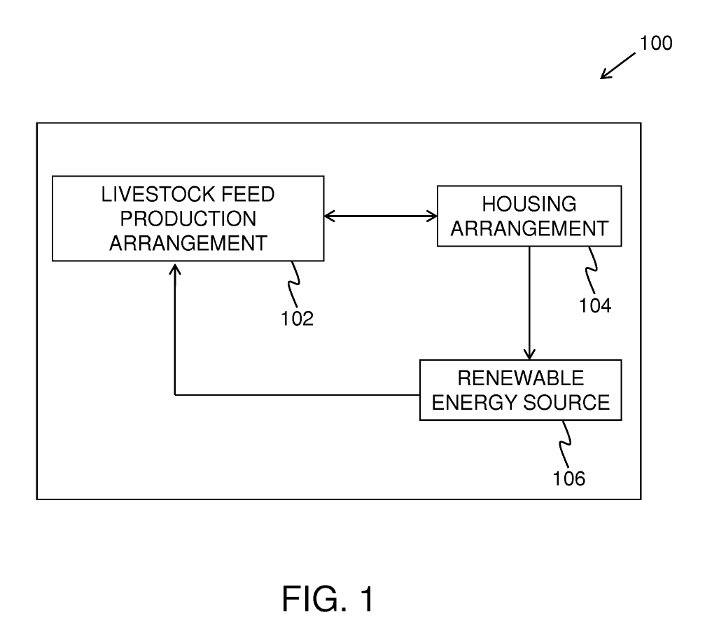

FIG. 1 is a block diagram of an agricultural system, in accordance with an embodiment of the present disclosure;

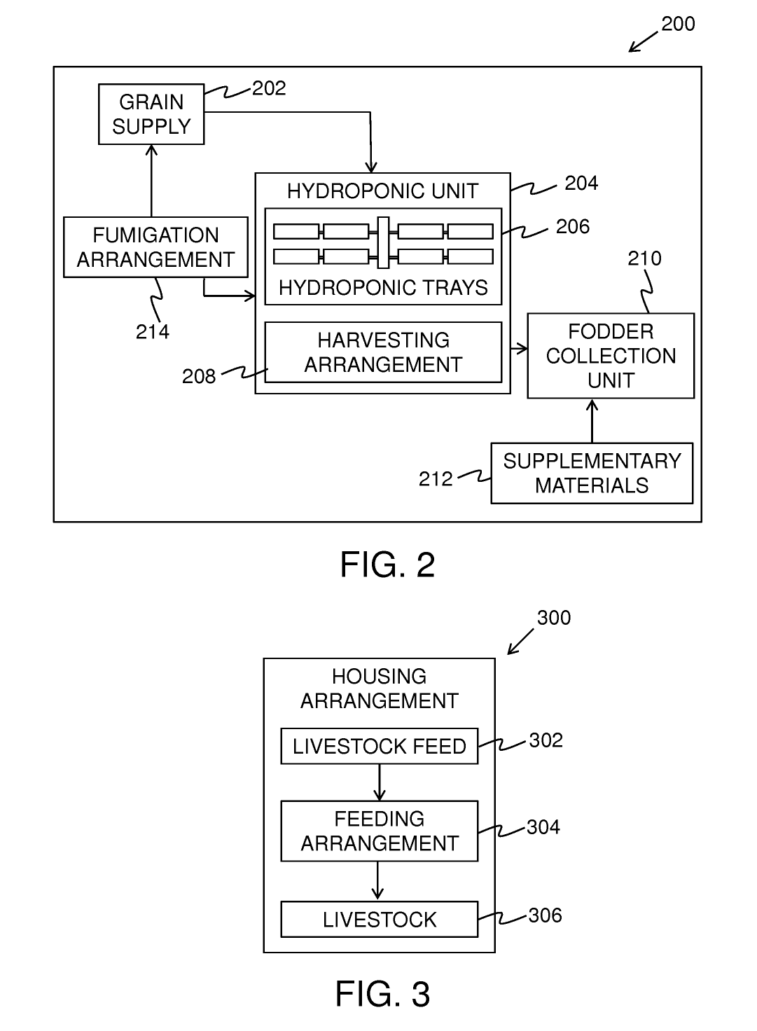

FIG. 2 is a block diagram of a livestock feed production arrangement of the agricultural system of FIG. 1, in accordance with an embodiment of the present disclosure;

FIG. 3 is a block diagram of a housing arrangement of the agricultural system of FIG. 1, in accordance with an embodiment of the present disclosure;

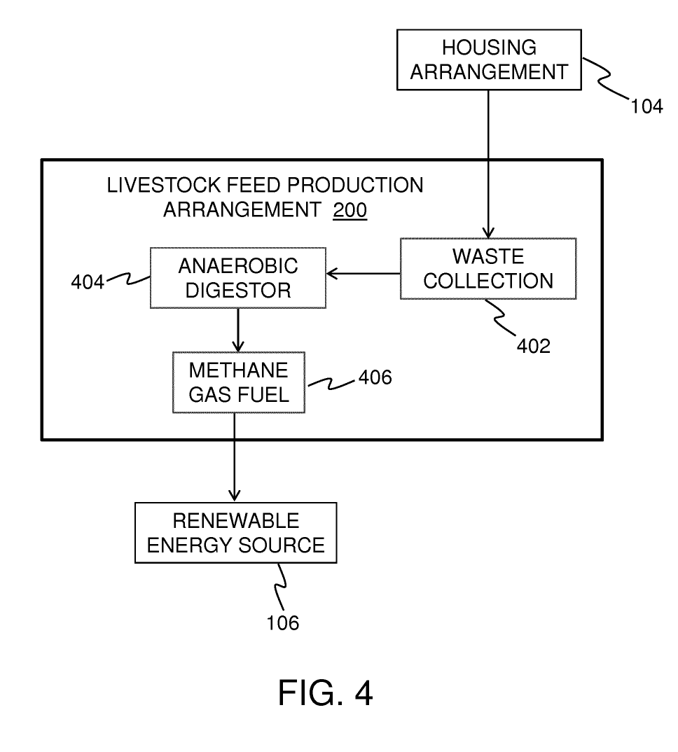

FIG. 4 is a block diagram of additional components of the livestock feed production arrangement of FIG. 2, in accordance with an embodiment of the present disclosure; and

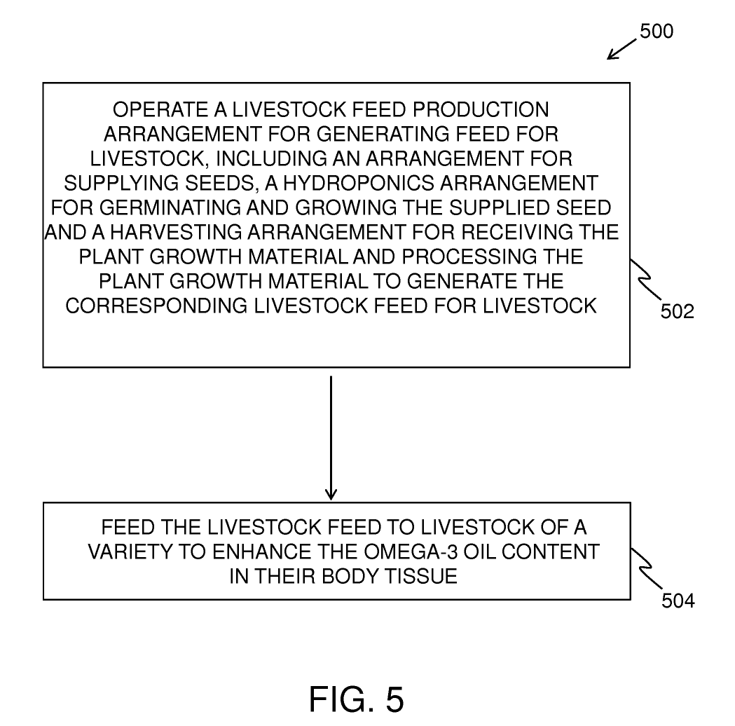

FIG. 5 is an illustration of steps of a method for enhancing Omega-3 oil content in body tissue of livestock, in accordance with an embodiment of the present disclosure.

In the accompanying diagrams, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DESCRIPTION OF EMBODIMENTS

FIG. 1 is a block diagram of an agricultural system 100, in accordance with an embodiment of the present disclosure. Specifically, FIG. 1 illustrates various functional components associated with the agricultural system 100 that enables in enhancing Omega-3 oil content in livestock. The agricultural system 100 includes a livestock feed production arrangement 102 for producing livestock feed for the livestock and processing waste from the livestock. The agricultural system 100 also includes a housing arrangement 104 for accommodating livestock. Further, the livestock is fed in operation from the livestock feed production arrangement 102. The agricultural system 100 also includes a renewable energy source 106 operatively coupled to the both livestock feed production arrangement 102 and the housing arrangement 104. For example, the energy from a methane gas fuel, generated from the waste obtained from the livestock accommodating in the housing arrangement 104, may be used to power the renewable energy source 106 which in turn may power the livestock feed production arrangement 102. Additionally, the renewable energy source 106 may be operable to employ off-grid with electricity supplied from at least one of: solar cells, wind turbines, naturally-occurring water flow such as hydroelectric energy, tidal energy and so forth.

FIG. 2 is a block diagram of a livestock feed production arrangement 200 of the agricultural system 100 of FIG. 1, in accordance with an embodiment of the present disclosure. Specifically, FIG. 2 illustrates various functional components of the livestock feed production arrangement 200 for generating livestock feed. The livestock feed production arrangement 200 includes a grain supplying arrangement 202 for supplying grains and a hydroponic arrangement 204 for receiving the grains from the grain supplying arrangement 202, and generating livestock feed for livestock corresponding to plant growth material in the hydroponic arrangement 204. The hydroponic arrangement 204 further include one or more hydroponic trays 206 for germinating supplied grain received from the grain supplying arrangement 202, and a harvesting arrangement 208 for receiving the plant growth from the hydroponic trays 206. Further, the livestock feed production apparatus includes a fodder collection unit 210 for blending the plant growth received from the harvesting arrangement 208 and supplementary minerals 212 to generate livestock feed.

In an embodiment, the supplied grain from grain supply unit 202 may be one of Spring Barley, although other types of grain such as alfalfa, maize, oats can also be used for fodder production. The supplied grain may be grown in the hydroponic trays 206 in a light-excluded environment (for example, dark arrangement) to provide for accelerated grain germination. Growth in the dark environment causes the grain to metabolize carbohydrate sugars present in the grain, reacting with gaseous oxygen, together with water and nutrients in the hydroponic solution to provide a nourishing biological growth that is pulverized to provide feed to the livestock.

In an embodiment, the fodder collection unit 210 may blend the plant growth material received from the harvesting arrangement 208 with supplementary materials 212 required for increasing the roughness of the fodder, resulting in a fibrous livestock feed. The supplementary materials 212 may be blended in a temporally varying manner with the plant growth material received from the harvesting arrangement 208.

In an embodiment, the supplementary materials 212 may include one of hay, wild flower supplements, aromatic supplements, vitamin supplements, growth supplements, antibiotics, and so forth.

In an embodiment, the method includes a fumigation arrangement 214 of the hydroponic arrangement 204, operable to remove moulds on the supplied grain by gaseous fumigation during germination of the grain in light-excluded environment. In present embodiment, in fumigation arrangement 214 may use ozone gas for fumigating the supplied grain to reduce growth of mould in the plant growth material. The ozone gas may be used temporally periodically in combination with sulphur dioxide and/or chlorine gas for fumigating the supplied grain in response to detection of mould formation upon the supplied grain, its roots and/or shoots.

In an embodiment, formation of Aspergillus on the supplied grains may be reduced by fumigating the germination and growth trays by ozone gas. For example, the germination and growth trays may be exposed to the ozone gas in periodic intervals. The ozone gas concentration may be in range of 20 parts per million (ppm) to 40 parts per million (ppm). The ozone exposure may be implemented for a duration of 5 minutes to 60 minutes (1 hour). The ozone is beneficially generated from a discharge process, for example from a corona discharge process or similar. The ozone is preferably directed to roots of the germinated grain in the trays, where possible. The grain is spread evenly around the trays to avoid bunching of distribution of the grain that could cause potential occurrence of Aspergillus or similar mould growth.

In another embodiment, formation of Aspergillus on the roots of the plant growth material may be further reduced by exposing to the ozone. For example, at periodic intervals, the hydroponic trays 206 may be exposed to ozone gas (via gaseous fumigation). Optionally, the grains received from the grain supplying arrangement 202 may be treated only once with ozone immediately before germination, but not later during its growing phase. Optionally, ozone treatment is applied in an event, during the growing phase, that Aspergillus is detected.

Optionally in an embodiment, other gases are employed in combination with ozone, for example Sulphur Dioxide, Nitrous Oxides, to hinder mould growth, with periodic application as aforementioned. Alternatively, the gaseous treatment for Aspergillus is supplied continuously. Alternatively, the gaseous treatment for Aspergillus is selectively applied only when a trace of Aspergillus is first detected. The trays are beneficially treated with ozone and/or bleach to kill any residual Aspergillus or similar mould between growth cycles of the grain therein.

In another embodiment, the ozone may be generated from a discharge process, for example from a corona discharge process, ultraviolet light exposure process or similar. The ozone is preferably directed to roots of the germinated grain in the hydroponic trays 206.

In an embodiment, the supplied grain may be spread evenly around the trays for avoiding bunching of distribution of the grain that may cause potential occurrence of Aspergillus or similar mould growth.

In an embodiment, the method includes arranging for the plurality of hydroponic trays 206 to have a length-to-width ratio in a range of 3:1 to 10:1.

In another embodiment, the method includes arranging the hydroponic trays 206 of the hydroponic arrangement 204 in a vertical manner on the actuation arrangement for preventing ponding along the trays. For example, actuation arrangement may be activated in a sequence along a length of the trays to sweep any ponding along the trays. The actuators may be electromagnetic actuators that are actuated under computer control, although hydraulic actuators can alternatively be employed.

In an embodiment, the plurality of hydroponic trays 206 may be mounted upon a moveable carousel for allowing automated loading of the seeds onto the plurality of trays and automated off-loading of their germinated seeds, and their roots and shoots, onto a carrier for removal from the hydroponics arrangement 204 for feeding to livestock.

FIG. 3 is a block diagram of a housing arrangement 300 of the agricultural system 100 of FIG. 1, in accordance with an embodiment of the present disclosure. Specifically, FIG. 3 illustrates various functional components of the housing arrangement 300 for accommodating livestock. For example, the livestock is fed in operation from the livestock feed production arrangement 102 (shown in FIG. 2). More specifically, the housing arrangement 300 includes a livestock feed unit 302 for receiving the processed livestock feed from the fodder production unit 210 (shown in FIG. 2). The housing arrangement 300 further includes a feeding arrangement 304 for feeding the livestock feed to livestock 306 of a variety to enhance the Omega-3 oil content in their body tissue.

In an embodiment, the housing arrangement 300 may be a closed structure for accommodating the livestock 306 therein. Further, the housing arrangement 300 may be provided with environmental controls of the system, to regulate the parameters such as temperature and aeration, along with sufficient space and ventilation in order to promote welfare of the livestock housed in the compound. In addition, the housing arrangement 300 may have an effective drainage system for the proper removal of excreta and trash. The waste removed from the housing arrangement 300 may be sent to a waste collection unit for the digestion of the waste in an anaerobic digester, which is explained herein later in conjunction with FIG. 4.

In an embodiment, the livestock 306 comprises of a Piedmontese variety. The Piedmontese variety of cattle is particularly effective at converting the feed to Omega-3 oils in their meat. The livestock, other than being of Piedmontese variety could comprise of other breeds with an enhanced amount of Omega-3 oil content in the body tissue, for example shorthorn and Jersey cattle. In addition, the livestock may include other categories other than cattle such as poultry, sheep, goats, camels, horses, pigs and so forth.

FIG. 4 is a block diagram of additional components of the livestock feed production arrangement 200 of FIG. 2, in accordance with an embodiment of the present disclosure. Specifically, FIG. 4 illustrates various functional components for generating methane gas from the anaerobic digestion of the waste product received from the housing arrangement 104. As shown, the livestock feed production apparatus 200 additionally includes a waste collection arrangement 402 for receiving waste from the livestock 306 (shown in FIG. 3) of the housing arrangement 104. Further, the livestock feed production apparatus 200 includes an anaerobic digester 404 for digesting the waste received from waste collection arrangement 402 to produce methane gas fuel 406. Moreover, the renewable energy source 106, is shown to receive the methane gas fuel 406, to generate electrical energy to be further supplied to the livestock feed production arrangement 102.

FIG. 5 is an illustration of steps of a method 500 for enhancing Omega-3 oil content in body tissue of livestock, in accordance with an embodiment of the present disclosure. Those skilled in the art would recognize that the method 500 illustrates steps involved in implementing the agricultural system 100, explained in conjunction with FIGS. 1-4.

At step 502, a livestock feed production arrangement is operated for generating feed for livestock. The livestock feed production arrangement includes an arrangement for supplying seeds, a hydroponics arrangement for receiving the supplied seeds and for germinating and growing the supplied seed in a light-excluded environment in one or more hydroponics trays to generirate plant growth material, and a harvesting arrangement for receiving the plant growth material and processing the plant growth material to generate the corresponding livestock feed for livestock.

At step 504, the livestock feed is fed to livestock, of a variety, to enhance the Omega-3 oil content in their body tissue.

The method for enhancing Omega-3 oil content in body tissue of livestock provided in the present disclosure may include operating a livestock feed production arrangement configured to maintaining optimum level of CO₂ required for photosynthesis of germinated seeds or fodder. For example, a gas feed arrangement may include one or more sensors for measuring the levels of CO₂ and/or for controlling the release of CO₂, maintaining it a required level or an optimum level of CO₂. The level of CO₂ may be controlled by a gas feed arrangement, or by adjusting one or more parameters of the fodder growth process, for example changes in temperature of operation which would with speed or slow chemical (or bio-chemical) reactions, including the fodder growth rate.

The steps 502 to 504 are only illustrative and other alternatives can also be provided where one or more steps are added, one or more steps are removed, or one or more steps are provided in a different sequence without departing from the scope of the claims herein. For example, the method 500 also includes using techniques, such as artificial insemination or embryo implanting, to arrange for the cattle to undergo 3 pregnancies within an approximate 2-year period, or a period between 2 and 3 years, depending on contextual conditions. The cattle may be fed with a varying diet of supplementary materials blended with fodder, which are tailored according to the needs of the cattle. The cattle may need higher concentrations of proteins in their diet during the breeding and the gestation periods and the supplementary materials may be added accordingly.

Modifications to embodiments of the invention described in the foregoing are possible without departing from the scope of the invention as defined by the accompanying claims. Expressions such as “including”, “comprising”, “incorporating”, “consisting of”, “have”, “is” used to describe and claim the present invention are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

1. A method for enhancing Omega-3 oil content in body tissue of livestock, wherein the method comprises:
(i) operating a livestock feed production arrangement for generating feed for livestock, wherein the livestock feed production arrangement includes an arrangement for supplying seeds, a hydroponics arrangement for receiving the supplied seeds and for germinating and growing the supplied seed in a light-excluded environment in one or more hydroponics trays to generate plant growth material, and a harvesting arrangement for receiving the plant growth material and processing the plant growth material to generate the corresponding livestock feed for livestock; and
(ii) feeding the livestock feed to livestock of a variety to enhance the Omega-3 oil content in their body tissue.

2. A method of claim 1, wherein the variety of the livestock comprises Piedmontese.

3. A method of claim 1, wherein the method includes arranging for the cattle to undergo 3 pregnancies within a 2-year period by use of artificial insemination, embryo implanting and varying a diet of the plant growth material in coordination with the pregnancies.

4. A method of claim 1, wherein the method includes operating the livestock feed production arrangement to blend supplementary materials into the corresponding livestock feed.

5. A method of claim 4, wherein the livestock feed production arrangement is operable to blend the supplementary materials into the corresponding livestock feed in a temporally varying manner.

6. A method of claim 3, wherein the method includes arranging for the supplementary materials to include at least one of: hay, wild flower supplements, aromatic supplements, vitamin supplements, growth supplements, antibiotics.

7. A method of claim 1, wherein the method includes employing a gaseous fumigation arrangement for gaseously fumigating the supplied seed prior to germination to reduce an occurrence of mould growth therein during germination and growing of the seeds to form roots and shoots.

8. A method of claim 7, wherein the method includes operating the gaseous fumigation arrangement to employ ozone gas for fumigating the supplied grain to reduce growth of mould in the plant growth material.

9. A method of claim 7, wherein the method includes providing the ozone gas for fumigation in a concentration in a range of 20 to 40 part per million for a period in a range of 5 minutes to 1 hours.

10. A method of claim 8, wherein the method includes providing the ozone gas for fumigation temporally periodically for fumigating the supplied grain in response to detection of mould formation upon the supplied seeds, their roots and/or their shoots.

11. A method of claim 8, wherein the method includes applying the ozone gas in combination with sulphur dioxide and/or chlorine gas.

12. A method of claim 1, wherein the method includes arranging for the one of more hydroponics trays have a length:width ratio in a range of 3:1 to 10:1.

13. A method of claim 1, wherein the method includes arranging for the one or more hydroponics trays to be provided with an actuation arrangement for selectively flexing regions of the one or more hydroponics trays for sweeping ponding of nutrient solution occurring therein along a length of the one or more hydroponics trays.

14. A method of claim 1, wherein the method includes arranging for the one or more hydroponics trays to be disposed in operation on a carousel for at least one of: applying seeds to the one or more hydroponics trays for growing the plant growth material on the one or more hydroponics trays, harvesting the plant growth material from the one or more hydroponics trays.

15. A method of claim 1, wherein the method includes arranging for the one or more hydroponics trays to be disposed in one or more vertical stacks, wherein planes of the one or more hydroponics trays for receiving the grain are mutually parallel.

16. An agricultural system includes the livestock feed production arrangement and a housing arrangement for accommodating livestock, wherein the livestock is fed in operation from the livestock feed production arrangement, wherein the livestock feed production arrangement is operated according to a method of claim 1.

17. An agricultural system of claim 16, wherein the livestock feed production apparatus includes an arrangement for receiving waste from the animals, and for anaerobically digesting the waste to generate methane gas.

18. An agricultural system of claim 16, wherein the agricultural system is operable off-grid with electricity supplied from at least one of: methane fuel, solar cells, wind turbines, naturally-occurring water flow.

19. A computer program product comprising a non-transitory computer-readable storage medium having computer-readable instructions stored thereon, the computer-readable instructions being executable by a computerized device comprising processing hardware to execute a method of claim 1.
